(19) United States
(12) Patent Application Publication
(10) Pub. No.: US 2011/0117208 A1
(43) Pub. Date: May 19, 2011

(54) CHRONIC WOUND TREATMENT

(75) Inventors: Johnathan Craig Richardson, Notts (GB); Peter William Dettmar, East Yorkshire (GB); Rebecca Louise Allen, Sheffield (GB); Cathal Padraig Coyle, Hull (GB)

(73) Assignee: TECHNOSTICS LIMITED, Hull (GB)

(21) Appl. No.: 12/918,826

(22) PCT Filed: Feb. 23, 2009
(86) PCT No.: PCT/GB09/00504

§ 371 Date: Dec. 7, 2010

(30) Foreign Application Priority Data
Feb. 22, 2008 (GB) 0803284.9
Feb. 22, 2009 (GB) 0803283.1

(51) Int. Cl. A61K 33/42; A61P 17/02

(52) U.S. Cl. 424/606; 424/601

(57) ABSTRACT

The invention provides the use of an unbound polyphosphate to promote chronic wound healing, in which the polyphosphate has at least 3 phosphate units. Also provided are a method of treatment and a pharmaceutical composition, both based on the use of the polyphosphate.

### FIELD OF THE INVENTION

The present invention is concerned with chronic wound healing and, in that connection, includes the use of certain compounds, as well as methods of treatment and pharmaceutical compositions.

### BACKGROUND TO THE INVENTION

Wound healing is a complex physiological process; however, it normally follows a predictable ordered sequence of events. This sequence has been arbitrarily divided into a series of stages namely (i) an inflammatory phase; (ii) a tissue formation phase and (iii) a tissue regeneration phase. Acute wounds, which are often caused by trauma/surgery, will repair by progressing through these stages in an ordered and timely manner. In contrast, chronic wounds do not follow this ordered sequence and become ‘stuck’ in the inflammatory phase. This delays the healing process and means certain chronic wounds may never heal or may take years to do so (Rovee DT and Maibach HI in The Epidermis in Wound Healing (2003) Informa HealthCare, NY, US).

It has been suggested that chronic wounds do not follow the sequence of healing stages that occurs with acute wounds because the spatial and temporal expression of degradative enzymes has been distorted. In the early stages of healing, enzymes are specifically expressed to degrade extracellular matrix (ECM). ECM is the component of tissue that, amongst other roles, provides support and anchorage for cells. In broad terms there are three major components of ECM: fibrous elements (e.g. collagen, elastin), link proteins (e.g. fibronectin) and space filling molecules (e.g. hyaluronan). The degradation of ECM during healing allows cells such as fibroblasts, neutrophils and macrophages to migrate into the wound area to effect the tissue repair. ECM degradation is also essential to allow the ECM to remodel so that it can support the synthesis of new tissue. As the healing process progresses the activity of ECM degrading enzymes in wounds should be reduced to allow a balance between the necessary breakdown and restructuring of existing ECM and the synthesis of new ECM. The persistent activity of degrading enzymes at the site of tissue repair may explain the chronicity of wound healing. For example, studies by numerous researchers have demonstrated that the levels of enzymes that degrade ECM are considerably higher in chronic wounds than in acute wounds. Yager et al (J Invest Dermatol 1996; 107: 743-8) showed that levels of MMP-2 and MMP-9 were significantly higher in wound fluid from chronic pressure ulcers compared to acute surgical wounds. Yager et al (Wound Rep Reg 1997; 5: 23-32) also showed that chronic wound fluids had elevated levels of elastase activity. Finally, Dechert et al (Wound Rep Reg 2006; 14: 252-258) showed that hyaluronidase activity in pressure ulcers was significantly elevated compared with acute wounds.

The differing biochemical composition of the wound microenvironment in chronic wounds compared to acute wounds means that the methods of treating these distinct wound types is different. Falanga (Wounds 2002; 14 (2): 47-57) states that using therapeutic agents and wound bed preparation methods suitable for acute wounds are not appropriate for chronic wounds. This is because chronic wounds do not follow the ordered healing stages of acute wounds.

Chronic wounds may occur in humans both internally and externally. Internal chronic wounds result from damage to the epithelium of the gastrointestinal tract and can occur as lesions or ulcerations in the oral cavity, throat, oesophagus, stomach, small and large intestine, colon and rectum. External chronic wounds affect the epidermis/dermis and include, for example, diabetic foot ulcers, venous stasis ulcers and pressure ulcers.

The healing of internal wounds is complicated by the presence of other proteolytic enzymes in the gastric intestinal tract. Gastric juice contains a series of aspartic proteases (Pepsin 1, 3a, 3b, 3c and gastricsin), which are synthesised in the gastric mucosa as an inactive precursor (pepsinogen) and, following stimulation of gastric chief cells, are released into the gastric lumen where they are activated by hydrochloric acid. The primary function of pepsin is to degrade dietary proteins and peptides into amino acid fragments suitable for absorption. Pepsin does not specifically degrade dietary protein and will indiscriminately cleave any suitable protein, peptide or glycoprotein. It will therefore degrade a range of constitutive proteins which are essential for normal physiological function, such as collagen and elastin. Indiscriminate degradation of these proteins, sometimes called autodigestion, is the underlying pathology of a number of disease states including dyspepsia, gastritis, ulceration and gastroesophageal reflux disease. In these disease states, the mucosa of the gastrointestinal tract is damaged by the proteolytic activity of pepsin.

The gastric mucosa is protected from pepsin degradation by a number of defence mechanisms including the secretion of a mucus gel layer. The mucus gel layer acts as a diffusion barrier to prevent an interaction between pepsin and the underlying mucosal surface proteins. The mucus layer can, however, be degraded by pepsin and therefore a dynamic balance exists between mucus secretion and degradation. If this balance is disturbed, and the mucus barrier compromised, pepsin can digest the underlying epithelium and collagen resulting in tissue destruction and gastric injury. Similarly, if pepsin is refluxed beyond the oesophageal sphincter into the aero-digestive tract, extensive tissue damage can occur as the epithelium above this sphincter does not possess the protective mechanisms present in the stomach. The reflux of gastric contents into the oesophagus and larynx may give rise to damage to the squamous epithelium of the oesophagus and larynx and may cause gastroesophageal reflux disease and extra-esophageal reflux disease as well as predisposing the mucosa to Barrett's oesophagus and oesophageal or laryngeal carcinoma.

Excess activity of enzymes which degrade ECM (including collagenase, hyaluronidase and elastase) contribute to the chronicity of both internal chronic wounds, such as lesions/ulceration of the gastrointestinal tract and external chronic wounds, such as diabetic foot ulcers, venous stasis ulcers and pressure ulcers. Furthermore, the proteolytic enzyme pepsin is implicated as the causative enzyme in gastric, oesophageal and laryngeal lesions/ulceration as well as contributing to their delayed healing.

In addition, enzymes which degrade ECM and structural biomolecules in skin (including collagenase, hyaluronidase and elastase) have been implicated as a causative factor in the onset and progression of skin ageing.

The inhibition of such enzymes or the protection of the matrix components from degradation has been identified as a point of intervention in associated disorders. Thus, the regulation and/or inhibition of ECM active enzymes, for example aspartic proteinases, such as pepsin, matrix metalloproteinases (MMPs), such as collagenase, serine proteinases, such as elastase, and/or glycoside hydrolases, such as hyaluronidase, may be advantageous in the repair and/or maintenance of a robust ECM.

Polyphosphates are generally linear polymers of many tens or hundreds of orthophosphate residues linked by high-energy, phosphoanhydride bonds.

Polyphosphate is found in a broad spectrum of living cells and one of its roles is believed to be to serve as a phosphate storage reservoir for the production of ATP (adenosine triphosphate), which provides the energy to power a cell. Recently, it has been disclosed that in bacteria, polyphosphate helps these single-celled organisms adapt to nutritional deficiencies and environmental stresses. For example, when bacteria are subjected to nutritional deficiencies or environmental stresses (e.g., heat or osmotic pressure), polyphosphate is synthesized to supply the energy necessary for the production of various proteins.

International Patent application No. WO 01/72313, from Kyung Won Medical, describes the use of various polyphosphates (P₃ to P₇₉₈) in promoting wound healing and scar abatement of acute wounds.

U.S. Pat. No. 6,599,523 which describes the use of a phosphorylated wound dressing, formed from a 4 to 16% composition of sodium hexametaphosphate for the treatment of chronic, non-healing wounds. The wound dressings are composed of a support matrix, such as cotton cellulose, and an active agent associated with the support matrix. The active agent may be a protease sequestrant, in particular a sequestrant of a neutrophil-derived cationic protease such as elastase.

More recently, Edwards, et al, Journal of Biomedical Materials research Part A DOI 10.1002, 446-454; describes the use of phosphorylated cotton dressings in sequestration of elastase and collagenase. However, the prior art compositions act as sequestration agents and thereby require significantly higher phosphate content.

### STATEMENTS OF THE INVENTION

According to the present invention, there is provided the use of an unbound polyphosphate to promote chronic wound healing, in which the polyphosphate has at least 3 phosphate units.

By “unbound” there is meant that the polyphosphate is not chemically bound to any other compound or substrate when it is in active use in wound treatment. Although it may be chemically bound as stored or even as applied, it becomes “unattached” under the treatment conditions. It may be physically bound to, for instance, a substrate carrier but, again, it is essentially behaves as free polyphosphate during treatment.

The polyphosphates of use in the present invention may be administered, for instance, externally, topically or enterally and may be effective in inhibiting various enzymes including pepsin and those involved in the restructuring of the ECM, such as collagenase, elastase and hyaluronidase. Chronic conditions which are associated with excessive activity of these enzymes are:

Chronic external wounds, such as pressure ulcers, diabetic ulcers etc—the chronicity of these wounds result from excessive activity of collagenase (MMPs), hyaluronidase and elastase; and
Chronic internal wounds, such as lesions/ulceration of the GI tract—enzymes that degrade ECM are responsible for causing these internal wounds (pepsin) and for their chronicity (pepsin, collagenase, hyaluronidase and elastase).

Accordingly, it is preferred that the polyphosphate inhibits the action of at least one enzyme which contributes to the prevention or delay of wound healing. More preferably, the enzyme is one or more of pepsin, collagenase, elastase and hyaluronidase.

The polyphosphate may be a single chemical entity or it may be a mixture of polyphosphates with different numbers of phosphate units. Where it is a mixture, the average number of phosphate units is at least 3. Preferably, the polyphosphate has an average of from 4 to 40 phosphate units.

Preferably, the polyphosphate has a P₂O₅ content of at least 55% by weight, more preferably at least 60% by weight. A preferred range is 60 to 75% by weight, more preferably 65 to 70% by weight.

By way of example, the material known as sodium hexametaphosphate is a mixture of polyphosphates with an average of 12 phosphate units and a P₂O₅ content of about 68%.

Preferably, the polyphosphate is an alkali metal salt, more preferably a sodium or potassium salt, or a mixture thereof.

The present invention also provides a method of promoting chronic wound healing comprising the administration of a therapeutically effective amount of a polyphosphate having at least 3 phosphate units and/or as indicated above.

Preferably the amount of polyphosphate is from 300 mg to 24,000 mg as a daily dosage.

Preferably, the polyphosphate is administered at a pH of from 2 to 6.

The present invention further provides a pharmaceutical composition comprising a therapeutically effective amount of a polyphosphate having at least 3 phosphate units in admixture with a pharmaceutically acceptable adjuvant, diluent or carrier.

### MORE DETAILED STATEMENTS OF THE INVENTION

Examples of polyphosphates of use in the present invention are trimetaphosphate (P₃) and hexametaphosphate (ostensibly P₆ although in practice about P₁₂).

The polyphosphate may be, for instance, an alkali metal salt (as mentioned above), an alkaline earth metal salt, such as a calcium salt, or an ammonium salt. Preferably, it is a soluble salt.

The enzyme subject to inhibition by the polyphosphate may include an ECM active enzyme selected from the group consisting of a Matrix Metalloproteinase (MMP), a serine proteinase and a glycoside hydrolase. The MMP may be, for instance, collagenase, the serine proteinase may be, for instance, elastase and the glycoside hydrolase may be, for instance, hyaluronidase.

The one or more enzymes may be active in the upper digestive tract. They include an aspartyl proteinase, for instance, pepsin, and an extracellular matrix (ECM) enzyme, for instance, selected from the group consisting of a Matrix Metalloproteinase (MMP), a serine proteinase and a glycoside hydrolase. Examples are collagenase, elastase and a hyaluronidase.

The polyphosphate may be used in the manufacture of a medicament for the inhibition of an ECM or digestive tract active enzyme in a mammal.

The therapeutically effective amount of the polyphosphate may be from 0.1 to 500 mM and preferred ranges include 0.1 to 200 mM and 0.1 to 100 mM.

The polyphosphate may be administered at a pH, for instance, in the range of from 4 to 10. A preferred range includes 6 to 8 and a more preferred range is 6 to 7.

The method may be a method of reduction of scar formation and/or wound healing.

The method may be for the prevention, alleviation or treatment of gastric lesions caused by pepsin activity in a mammal. The therapeutically effective amount of the polyphosphate may be, for instance, from 0.1 to 500 mM or from 0.1 to 10 mM. The pH of administration may be in one of the ranges 2 to 8, 2 to 6 and 3 to 5.

The pharmaceutical composition may be in any suitable form including a dry powder, a gel or paste or as a liquid. The polyphosphate may be present in an amount of, for instance, 0.1 to 12.0% weight/volume. The composition may be in any suitable structure such as a spray, roll-on, patch, suspension, implant, sub-dermal depot, injection, lipstick/balm style applicator, suture/stitches or surgical glue.

The wound dressing for delivering a therapeutically effective amount of polyphosphate/trimetaphosphate to the wound site will contain a wound contacting material. The wound contacting material (incorporating the polyphosphate/trimetaphosphate) may take a number of forms. These include, without limitation, foams, fibers, fabrics, films, alginates, hydrogels, and hydrocolloids.

Foam generally refers to a cellular polymeric structure, and preferably an open cell structure. Suitable foams include such synthetic organic polymers as polyurethane, carboxylated butadiene styrene rubber, polyester, polyacrylate and non-synthetic/semi-synthetic polymers such as polysaccharides and there derivatives. It is generally desirable that the foam is hydrophilic; however, hydrophobic foams having a hydrophilic coating on them may be used. Hydrophilic foams include cellular polyurethane foam formed from isocyanates, polyether/polyester polyols and water, catalysts, stabilizers and other substances.

Fabric may be formed from fibers such as synthetic fibers, natural fibers, or combinations thereof. Synthetic fibers include, for example, polyester, acrylic, polyamide, polyolefin, polyaramid, polyurethane, regenerated cellulose, and blends thereof. More specifically, polyester includes, for example, polyethylene terephthalate, polytriphenylene terephthalate, polybutylene terephthalate, polylactic acid, and combinations thereof. Polyamide includes, for example, nylon 6, nylon 6,6, and combinations thereof. Polyolefin includes, for example, polypropylene, polyethylene, and combinations thereof. Polyaramid includes, for example, poly-p-phenyleneteraphthalamid (i.e., Kevlar®), poly-m-phenyleneteraphthalamid (i.e., Nomex®), and combinations thereof. Natural fibers include, for example, wool, cotton, flax, and blends thereof. The fabric may be of any variety, including but not limited to, woven fabric, knitted fabric, nonwoven fabric, or combinations thereof.

The film may include thermoplastic materials, thermoset materials, or combinations thereof. Thermoplastic or thermoset materials may include polyolefin, polyester, polyamide, polyurethane, acrylic, silicone, melamine compounds, polyvinyl acetate, polyvinyl alcohol, nitrile rubber, ionomers, polyvinyl chloride, polyvinylidene chloride, chloroisoprene, or combinations thereof. The polyolefin may be polyethylene, polypropylene, ethylvinyl acetate, ethylmethyl acetate, or combinations thereof. Polyethylene may include low density or high density polyethylene. The film may have a thickness of between about 1 and about 500 microns, or more preferably between about 1 and about 250 microns, or even more preferable between about 1 and about 100 microns.

Alginate is a natural polysaccharide that exists widely in many brown seaweeds. Sodium alginates are well known for their ability to form a gel in contact with most multivalent cations. Alginate fibers may be formed from alginate by extruding or spinning an alginate aqueous solution into a coagulating bath containing a multivalent cation (such as calcium) to cross-link and gel the alginate solution. The alginate fibers are then typically processed and incorporated into a wound care dressing.

Hydrogels generally consist of high-molecular molecules that form a coherent matrix for enclosing smaller molecules and aqueous solutions. Hydrogels can be described as a two-component system of water and a three-dimensional network polymer. Examples of hydrogels include starch, pectin, gelatin, other natural gums and insoluble cross-linked polymers such as polyethylene oxide.

Hydrocolloids are hydrophilic polymers, of vegetable, animal, microbial or synthetic origin, that generally contain many hydroxyl groups and may be polyelectrolytes. They are naturally present or added to control the functional properties of a material such as viscosity, including thickening and gelling, and water binding. They are advantageous for use as wound care devices because of their ability to absorb several times their weight in wound exudates. Examples of hydrocolloids include carbowax, vinyl polymers (such as polyvinyl alcohol, polyvinyl pyrrolidone, and polyvinylacetate), cellulose derivatives (such as ethyl cellulose, methyl cellulose, and carboxymethyl cellulose), and natural gums (such as guar, acacia, and pectins).

### DETAILED DESCRIPTION OF THE INVENTION

### Azocoll Dose Response Assay

Azocoll is a commercially available azo dye labelled collagen Type I substrate derived from bovine hide. In the presence of certain enzymes the red azo dye is liberated from the collagen and the resulting colour change can be measured and correlated with collagenolytic activity. The Azocoll assay was used to determine the inhibitory effect of polyphosphates on the action of pepsin, collagenase, snake venom metalloprotease, human gastric juice and human chronic wound fluid against azo-labelled collagen substrate.

### Materials

TABLE 1
Reagents and buffers used in the Azocoll assay. (All reagents were purchased
from standard laboratory suppliers, unless otherwise indicated)

| | |
|---|---|
| Pepsin solution | 100 μg/ml of Pepsin (from porcine gastric mucosa lyophilized |
| | powder, 3,200-4,500 units/mg protein: Sigma-Aldrich Ltd, UK) |
| | prepared in a buffered solution (pH 3-5 as desired) |
| Collagenase | 50 μg/ml of collagenase (crude collagenase secreted by Clostridium |
| solution | histolyticum, Sigma-Aldrich Ltd, UK) prepared in a buffered |
| | solution (pH 6-8 as desired) |
| Snake venom | 100 μg/ml of snake venom metalloproteinase (obtained from |
| metalloproteinase | Bothrops jararaca) prepared in a buffered solution (pH 7) |
| solution | |
| Human gastric | Human gastric juice (obtained by aspiration during endoscopy) was |
| juice | diluted with pH 4 buffer to a concentration that had a proteolytic |
| | activity equivalent to 100 μg/ml of porcine pepsin |
| Human chronic | Human chronic wound fluid was extracted from primary wound |
| wound fluid | dressing by soaking a single dressing overnight in 5 ml phosphate |
| | buffered saline. |
| pH 4.0-6.0 | 50 mM sodium acetate adjusted to relevant pH with glacial acetic |
| buffer | acid |
| pH 7.0-8.0 | 0.2M Tris(hydroxymethyl)aminomethane (Tris) corrected to |
| buffer | relevant pH using 0.2M hydrochloric acid |
| Azocoll solution | 0.30% w/v solution of Azocoll substrate (azo-dye labelled Type I |
| | collagen (Calbiochem, UK)) prepared in buffer |
| Inhibitor test | Solutions of the following test compounds were prepared in |
| solutions | deionised water at concentrations varying from 0-122 mg/ml |
| | Sodium polyphosphates containing: 60% P₂0₅; 65% P₂0₅; 68% P₂0₅; |
| | 69% P₂0₅; 70% P₂0₅ (Thermphos UK Ltd, UK and Fisher Scientific |
| | UK Ltd, UK) |
| | Potassium polyphosphate containing 60% P₂0₅ (Budenheim, |
| | Germany) |

### Method

A series of test solutions containing between 0-122 mg/ml of the proposed inhibitor were prepared. 100 μl of this test solution was then thoroughly mixed with 100 μl of the relevant enzyme solution on a vortex mixer before 1000 μl of buffered Azocoll solution was added and mixed. The inhibitor:enzyme:Azocoll sample was then incubated in a heated water bath for 3 hours at 37° C. and inverted every 30 minutes during this incubation time. Samples were then removed from the incubator, placed in iced water to cool and centrifuged (Fisher Scientific, accuSpin Model 400 Benchtop Centrifuge) at 4000 rpm for 5 minutes. The absorbance of the supernatant was measured at 540 nm (Labsystems Multiskan Ascent 354, ThermoFisher Scientific, Horsham, West Sussex, UK) using deionised water as a blank. The percentage inhibition of enzyme activity was calculated by comparing the absorbance intensity of test samples containing inhibitor with samples containing 0 μg/ml of inhibitor according to the equation below:

$$\% Inhibition = \frac{\begin{matrix}Standard ( 0 µg \text{/} ml inhibitor ) OD 540 nm - \\ Test OD 540 nm\end{matrix}}{Standard ( 0 mg \text{/} ml inhibitor ) OD 540 nm} \times 100$$

### Tricholoroacetic Acid (TCA) Precipitation Assay

The TCA assay is based on the method described by M. L. Anson (1938). J General Physiol, 22, 79-89. The substrate, bovine haemoglobin, is digested by pepsin. Any remaining undigested haemoglobin is then precipitated with TCA to yield a supernatant which contains only products of digested haemoglobin. The concentration of haemoglobin breakdown products in the supernatant is measured spectrophotometrically and provides an indication of proteinase activity. The TCA precipitation assay was used to determine the inhibitory effect of polyphosphates and trimetaphosphate on the action of pepsin against haemoglobin.

### Materials

TABLE 2
Reagents and buffers used in the trichloroacetic acid assay. (All reagents were
purchased from standard laboratory suppliers, unless otherwise indicated)

| | |
|---|---|
| Pepsin solution | 20 μg/ml of pepsin (from porcine gastric mucosa lyophilized |
| | powder, 3,200-4,500 units/mg protein: Sigma-Aldrich Ltd, UK) |
| | prepared in a buffered solution (pH 2-4 as desired) |
| pH 2.0 buffer | 0.2M glycine and 0.1M sodium chloride solution |
| Haemoglobin | 3.3 mg/ml solution of bovine haemoglobin (Sigma-Aldrich, UK)) |
| solution | prepared in relevant buffer |
| Trichloroacetic | 10% w/v solution of trichloroacetic acid prepared in deionised water |
| acid solution | |
| Inhibitor test | Solutions of the following test compounds were prepared in |
| solutions | deionised water at concentrations varying from 0-306 mg/ml |
| | Sodium polyphosphate containing: 68% P₂0₅ (Fisher Scientific UK |
| | Ltd) |
| | Sodium trimetaphosphate (Fisher Scientific UK Ltd, UK) |

### Method

A series of test solutions containing between 0-306 mg/ml of the proposed inhibitor were prepared. 100 μl of this test solution was then thoroughly mixed with 100 μl of pepsin solution on a vortex mixer before 1500 μl of buffered bovine haemoglobin solution was added and mixed. The inhibitor:enzyme:haemoglobin sample was then incubated in a heated water bath for 30 minutes at 37° C. The samples was then removed from the incubator, mixed with 2.0 ml TCA solution and left to stand for 30 minutes in iced water. The sample was then centrifuged (Fisher Scientific, accuSpin Model 400 Benchtop Centrifuge) at 4000 rpm for 5 minutes. The absorbance of the supernatant was measured at 280 nm (Labsystems Multiskan Ascent 354, ThermoFisher Scientific, Horsham, West Sussex, UK) using deionised water as a blank. The percentage inhibition of pepsin activity was calculated by comparing the absorbance intensity of test samples containing inhibitor with samples containing 0 μg/ml of inhibitor according to the equation below:

$$\% Inhibition = \frac{\begin{matrix}Standard ( 0 µg \text{/} ml inhibitor ) OD 280 nm - \\ Test OD 280 nm\end{matrix}}{Standard ( 0 mg \text{/} ml inhibitor ) OD 280 nm} \times 100$$

### Elastin Congo Red Assay

Elastin Congo red is a commercially available elastin substrate impregnated with the chromophore Congo red. In the presence of elastase the Congo red dye is liberated from the elastin and the resulting colour change can be measured and correlated with elastolytic activity. The elastin Congo red assay was used to determine the inhibitory effect of polyphosphates and trimetaphosphate on the elastase against the elastin Congo red substrate.

### Materials

TABLE 3
Reagents and buffers used in the elastin Congo red assay. (All reagents were
purchased from standard laboratory suppliers, unless otherwise indicated)

| | |
|---|---|
| Elastase | 1 in 30 dilution of Elastase (from porcine pancreas Type I, aqueous |
| solution | suspension, ≧4.0 units/mg protein: Sigma-Aldrich Ltd, UK) in |
| | relevant pH buffer (pH 7-8 as desired) |
| pH 7.0-8.0 | 0.2M Tris(hydroxymethyl)aminomethane (Tris) corrected to |
| buffer | relevant pH using 0.2M hydrochloric acid |
| Elastin Congo | 2.5 mg/ml Elastin Congo red (Sigma, UK) prepared in relevant pH |
| red solution | buffer (pH 7-8 as desired). |
| Inhibitor test | Solutions of the following test compounds were prepared in |
| solutions | deionised water at concentrations varying from 0-306 mg/ml |
| | Sodium polyphosphates containing: 60% P₂0₅; 65% P₂0₅; 68% P₂0₅; |
| | 69% P₂0₅; 70% P₂0₅(Thermphos UK Ltd, UK; Fisher Scientific UK |
| | Ltd, UK) |
| | Potassium polyphosphate: 60% P₂0₅ (Budhenheim, Germany) |
| | Sodium trimetaphosphate (Fisher Scientific UK Ltd, UK) |

### Method

A series of test solutions containing between 0-306 mg/ml of the proposed inhibitor were prepared. 100 μl of this test solution was then thoroughly mixed with 100 μl of elastase solution on a vortex mixer. 1000 W of buffered Elastin Congo red solution was then added and mixed. The inhibitor:enzyme:Elastin Congo red sample was then incubated overnight in a heated water bath at 37° C. Samples were then removed from the water bath and placed in iced water to cool for 30 minutes before being centrifuged (Fisher Scientific, accuSpin Model 400 Benchtop Centrifuge) at 13000 rpm for 5 minutes. The absorbance of the supernatant was measured at 540 nm (Labsystems Multiskan Ascent 354, ThermoFisher Scientific, Horsham, West Sussex, UK) using deionised water as a blank. The percentage inhibition of elastase activity was calculated by comparing the absorbance intensity of test samples containing inhibitor with samples containing 0 μg/ml of inhibitor according to the equation below:

$$\% Inhibition = \frac{\begin{matrix}Standard ( 0 µg \text{/} ml inhibitor ) OD 540 nm - \\ Test OD 540 nm\end{matrix}}{Standard ( 0 mg \text{/} ml inhibitor ) OD 540 nm} \times 100$$

### Hyaluronidase Dose Response Assay

The hyaluronidase activity assay is based on the methods of Bonner and Cantey (Clin. Chim. Acta, 13 (1966) 746-752) and Reissig et al. J. Biol. Chem., 217 (1955) 959-966. It relies on the fact that sodium hyaluronate is degraded in the presence of hyaluronidase into saccharides with N-acetylglucosamine (NAG) end-groups. The NAG can then be quantified by heating with alkaline tetraborate to form an intermediate which reacts with p-dimethylamino benzaldehyde in acidic medium to form a coloured product. The colour change can be measured and correlated with the activity of hyaluronidase. The hyaluronidase assay was used to determine the inhibitory effect of polyphosphates on the activity of hyaluronidase against the sodium hyaluronan substrate.

### Materials

TABLE 4
Reagents and buffers used in the hyaluronidase assay. (All reagents were
purchased from standard laboratory suppliers, unless otherwise indicated)

| | |
|---|---|
| Hyaluronidase | 1000 μg/ml mammalian hyaluronidase from bovine testes |
| solution | (Sigma, UK) in relevant buffer (pH 4-5 as desired). |
| pH 4-5 buffer | 50 mM sodium acetate adjusted with 50 mM acetic acid prepared |
| | from glacial acetic acid. |
| Sodium | 4 mg/ml sodium hyaluronate (Pharma Grade 80, 620-1200 kDa) |
| hyaluronate | (NovaMatrix, Drammen, Norway) prepared in relevant pH buffer. |
| solution | |
| Potassium | 0.8M Potassium tetraborate prepared in deionised water |
| tetraborate | |
| solution | |
| DMAB | p-dimethylamino benzaldehyde reagent (DMAB) (stock DMAB |
| solution | reagent: 10% w/v in 12.5% v/v concentrated hydrochloric acid in |
| | glacial acetic, this stock is diluted 1:10 in glacial acetic acid before |
| | use). |
| Inhibitor test | Solutions of the following test compounds were prepared in |
| solutions | deionised water at concentrations varying from 0-61.2 mg/ml |
| | Sodium polyphosphates containing: 60% P₂0₅; 65% P₂0₅; 68% P₂0₅; |
| | 69% P₂0₅; 70% P₂0₅ (Thermphos UK Ltd, UK; Fisher Scientific UK |
| | Ltd, UK) |
| | Potassium polyphosphate: 60% P₂0₅ (Budhenheim, Germany) |

A series of test solutions containing between 0-61.2 mg/ml of the proposed inhibitor were prepared. 100 μl of this test solution was then thoroughly mixed with 100 μl of 1 mg/ml hyaluronidase using a vortex mixer before 200 μl of 4 mg/ml buffered, sodium hyaluronate solution was added. The polymer:hyaluronidase:HA test sample was then thoroughly mixed using a vortex mixer and incubated at 37° C. for 4 hours. The reaction was subsequently terminated by heating at 80° C. for 5 mini After incubation, 60 μl of potassium tetraborate (0.8M) was added and the samples were again incubated at 80° C. for 5 minutes followed by cooling on ice for 5 minutes. Following cooling, 2.0 ml of DMAB solution was added and colour was allowed to develop at 37° C. for 20 minutes. Samples were then removed from the incubator and centrifuged (Fisher Scientific, accuSpin Model 400 Benchtop Centrifuge) at 1500 rpm (1854×g) for 10 mins. The absorbance of the supernatant was measured at 540 nm using deionised water as a blank. The percentage inhibition of hyaluronidase activity was calculated by comparing the absorbance intensity of test samples containing inhibitor with samples containing 0 μg/ml of inhibitor according to the following equation.

$$\% Inhibition = \frac{\begin{matrix}Standard ( 0 • 1 i nhibitor ) OD 540 nm - \\ Test OD 540 nm\end{matrix}}{Standard ( 0 • 1 inhibitor ) OD 540 nm} \times 100$$

### Lysozyme Dose Response Assay

The lysozyme dose response assay is based on the observation that in the presence of lysozyme the optical density of a cell suspension of Micrococcus lysodeikticus decreases.

The rate of this decrease in optical density can be measured and correlated with the activity of lysozyme. The lysozyme assay was used to determine the inhibitory effect of sodium polyphosphate (68% P₂O₅) on the action of lysosyme against the Micrococcus lysodeikticus cell suspension substrate.

### Materials:

TABLE 5
Reagents and buffers used in the lysozyme assay; (All reagents were purchased
from standard laboratory suppliers, unless otherwise indicated)

| | |
|---|---|
| Lysozyme | 50 μg/ml lysozyme from chicken egg white BioChemika, dialyzed, |
| solution | lyophilized, powder, ~100000 units/mg in pH 7 buffer |
| pH 7.0 buffer | 0.2M sodium phosphate corrected to pH 7 using 0.1M citric acid |
| Micrococcus | 0.015% w/v Micrococcus lysodeikticus cell suspension prepared in |
| lysodeikticus | buffer (Sigma-Aldrich Ltd, UK) |
| cell | |
| suspension | |
| Inhibitor test | Solutions of the following test compounds were prepared in |
| solutions | deionised water at concentrations varying from 0-61.2 mg/ml |
| | Sodium polyphosphates containing: 68% P₂0₅; (Thermphos UK Ltd, |
| | UK) |

### Method:

2.5 ml of Micrococcus lysodeikticus cell suspension was added to a cuvette (kartell disposable semi-micro PMMA, UV grade, 10 mm pathlength, 1.5 ml capacity, 280-800 nm range, ThermoFisher Scientific Horsham, West Sussex, UK) and placed in a thermostatically controlled UV spectrophotometer maintained at 25° C. 100 μl of test solution containing between 0-61.2 mg/ml of the proposed inhibitor was then added to the cuvette and thoroughly mixed by inversion. The inhibitor:substrate sample was then incubated at room temperature for 1 hour before 100 μl of lysozyme solution was added and mixed by inversion. The absorbance of the inhibitor:substrate:lysozyme test sample was measured at 450 nm at 3 second intervals for 5 minutes (Unicam UV 500, Thermo-Spectronic, Cambridge, UK). Buffer was used as a blank.

The inhibition of lysozyme activity was calculated by comparing the maximum linear gradient of the fall in optical density over the 5 minute period for test samples containing inhibitor with samples containing 0 μg/ml of inhibitor. The percentage inhibition was calculated according to the equation below:

$$\% Inhibition = \frac{\begin{matrix}Standard ( 0 µg \text{/} ml inhibitor ) Δ {A}_{450 nm} \text{/} min - \\ Test Δ {A}_{450 nm} \text{/} min\end{matrix}}{Standard ( 0 mg \text{/} ml inhibitor ) Δ {A}_{450 nm} \text{/} min} \times 100$$

### Chymotrypsin Dose Response Assay

The chymotrypsin dose response assay is based on the observation that in the presence of chymotrpysin the substrate Nₐ-benzoyl-L-tyrosine ethyl ester (BTEE) is degraded into Na-benzoyl-L-tyrosine+Ethanol. This conversion can be measured spectrophotometrically by an increase in absorbance at 253 nm. The rate of change in absorbance can then be measured and correlated with the activity of chymotrypsin. The chymotrypsin assay was used to determine the inhibitory effect of sodium polyphosphate (68% P₂O₅) on the action of chymotrypsin against the BTEE substrate.

### Materials:

TABLE 6
Reagents and buffers used in the chymotrypsin assay. (All reagents were
purchased from standard laboratory suppliers, unless otherwise indicated)

| | |
|---|---|
| Chymotrypsin | 100 μg/ml α-Chymotrypsin (from bovine pancreas Type II, |
| solution | lyophilized powder, ≧40 units/mg protein: Sigma-Aldrich Ltd, UK) |
| | in 1 mM hydrochloric acid |
| pH 7.8 buffer | 80 mM Tris HCl Buffer, pH 7.8 at 25° C. (Prepare 100 ml in |
| | deionized water using Trizma Base. Adjust to pH 7.8 at 25° C. with |
| | 1M HCl.) |
| N-Benzoyl-L- | 1.18 mM N-Benzoyl-L-Tyrosine Ethyl Ester (BTEE) |
| Tyrosine | (Prepare 50 ml using N-Benzoyl-L-Tyrosine Ethyl Ester (Sigma, |
| Ethyl Ester | UK) by initially dissolving in 31.7 ml of methanol and then |
| (BTEE) | correcting to a final volume of 50 ml using deionised water.) |
| solution | |
| Calcium | 2M calcium chloride solution in deionised water |
| chloride | |
| solution | |
| Inhibitor test | Solutions of the following test compounds were prepared in |
| solutions | deionised water at concentrations varying from 0-61.2 mg/ml |
| | Sodium polyphosphate containing: 68% P₂0₅; (Thermphos UK Ltd, |
| | UK) |

### Method:

1.28 ml of test solution containing between 0-61.2 mg/ml of the proposed inhibitor was added to a quartz cuvette and placed in a thermostatically controlled UV spectrophotometer maintained at 25° C. 1.26 ml of BTEE solution and 0.07 ml of calcium chloride solution were also added to the cuvette, mixed by inversion and allowed to equilibrate until the absorbance at 253 nm was constant. 0.09 ml of chymotrypsin solution was then added to the cuvette and immediately mixed by inversion. The absorbance of the inhibitor:substrate:chymotrypsin test sample was measured at 253 nm at 3 second intervals for 5 minutes (Unicam UV 500, Thermo Spectronic, Cambridge, UK). Buffer was used as a blank.

The inhibition of chymotrypsin activity was calculated by comparing the maximum linear gradient of the increase in absorbance at 253 nm over the 5 minute period for test samples containing inhibitor with samples containing 0 μg/ml of inhibitor. The percentage inhibition was calculated according to the equation below:

$$\% Inhibition = \frac{\begin{matrix}Standard ( 0 µg \text{/} ml inhibitor ) Δ {A}_{253 nm} \text{/} min - \\ Test Δ {A}_{253 nm} \text{/} min\end{matrix}}{Standard ( 0 mg \text{/} ml inhibitor ) Δ {A}_{253 nm} \text{/} min} \times 100$$

### Results

The following discussion makes reference to the accompanying drawings, which are:—

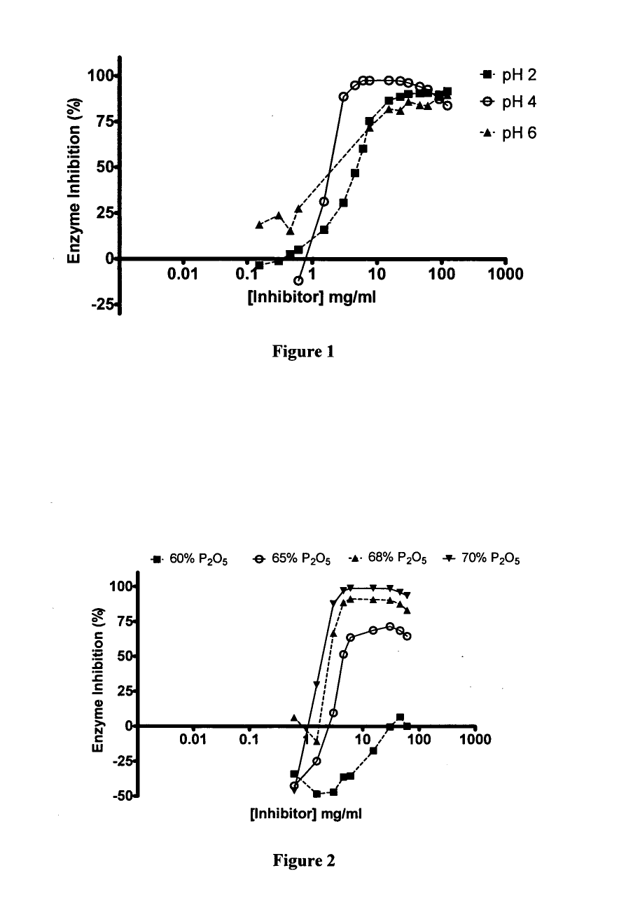

FIG. 1. Inhibition of pepsin by sodium polyphosphate (68% P₂O₅) at varying pH. Mean n=2 for pH2. Mean n=3 for pH 4 and 6. Errors bars omitted for clarity.

FIG. 2. Inhibition of pepsin by sodium polyphosphates varying in polymeric chain length (as defined by percentage P₂O₅ content) at pH 4. Mean n=1.

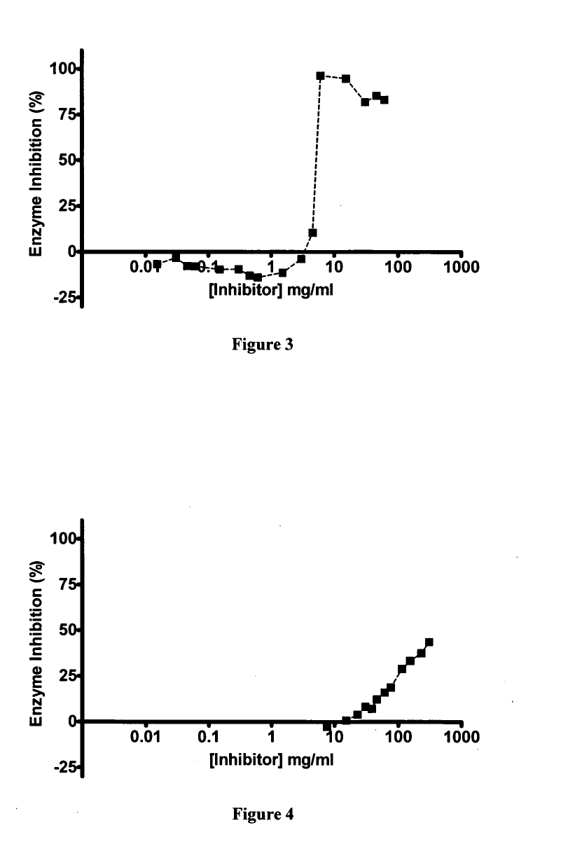

FIG. 3. Inhibition of pepsin by potassium polyphosphate (60% P₂O₅ content) at pH 4. Mean n=1.

FIG. 4. Inhibition of pepsin by sodium trimetaphosphate at pH 2. Mean n=1.

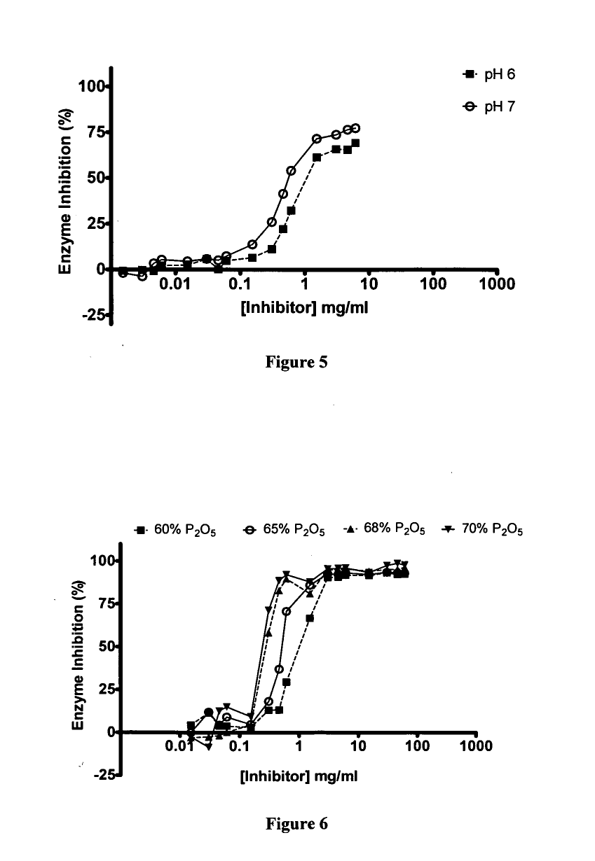

FIG. 5. Inhibition of collagenase by sodium polyphosphate (68% P₂O₅) at varying pH. Mean n=2. Errors bars omitted for clarity.

FIG. 6. Inhibition of collagenase by sodium polyphosphates varying in polymeric chain length (as defined by percentage P₂O₅ content) at pH 7. Mean n=1.

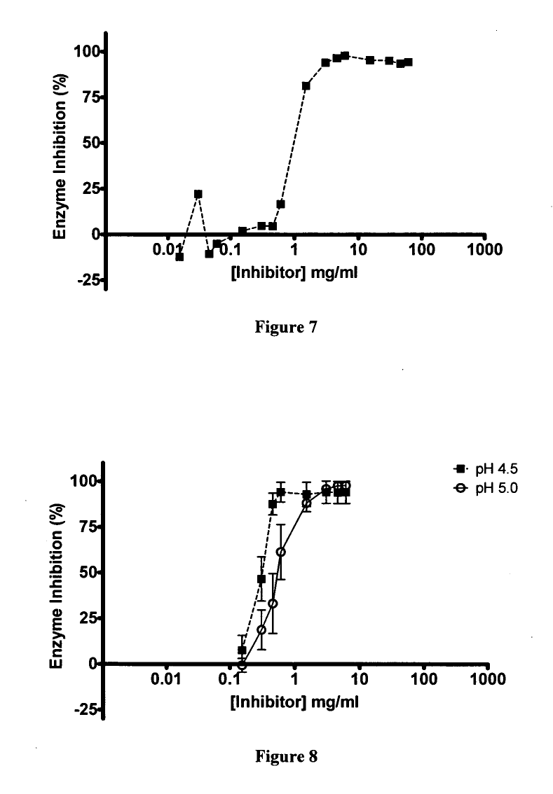

FIG. 7. Inhibition of collagenase by potassium polyphosphate (60% P₂O₅ content) at pH 7. Mean n=1.

FIG. 8. Inhibition of hyaluronidase by sodium polyphosphate (68% P₂O₅) at varying pH. Mean n=3±SEM.

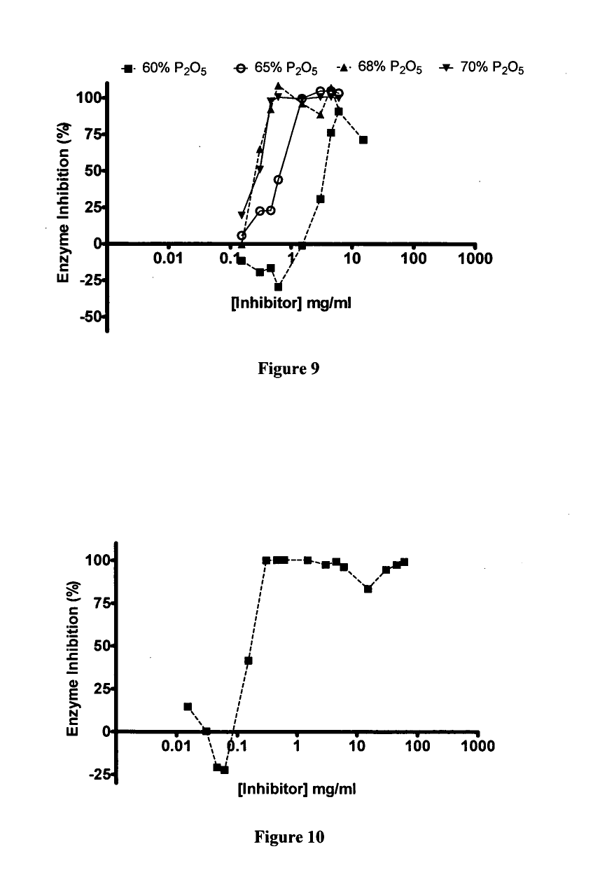

FIG. 9. Inhibition of hyaluronidase by sodium polyphosphates varying in polymeric chain length (as defined by percentage P₂O₅ content) at pH 4.5. Mean n=1.

FIG. 10. Inhibition of hyaluronidase by potassium polyphosphate (60% P₂O₅ content) at pH 7. Mean n=1.

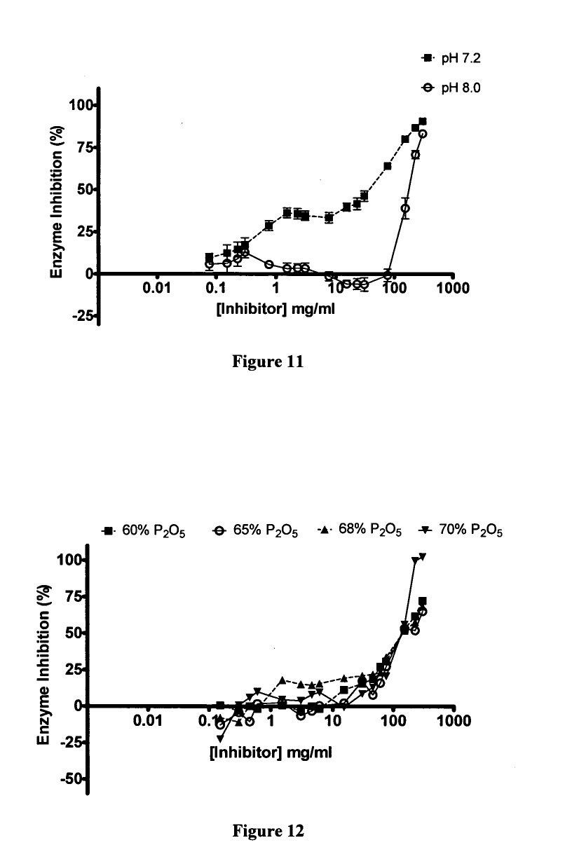

FIG. 11. Inhibition of elastase by sodium polyphosphate (68% P₂O₅) at varying pH. Mean n=3±SEM.

FIG. 12. Inhibition of elastase by sodium polyphosphates varying in polymeric chain length (as defined by percentage P₂O₅ content) at pH 8. All mean (n=1), except 68% P₂O₅ which is mean (n=4).

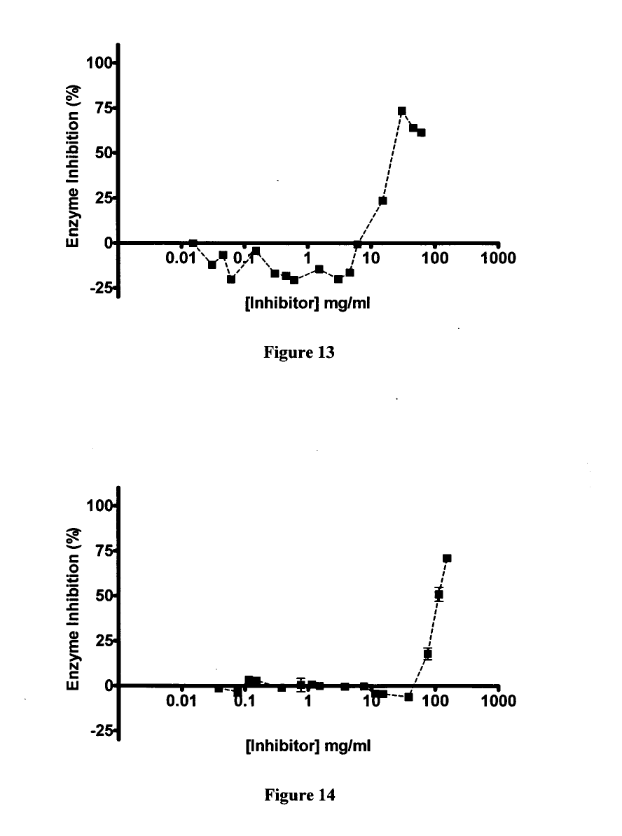

FIG. 13. Inhibition of elastase by potassium polyphosphate (60% P₂O₅ content) at pH 8. Mean n=1.

FIG. 14. Inhibition of elastase by sodium trimetaphosphate at pH 8. Mean (n=2)±SEM.

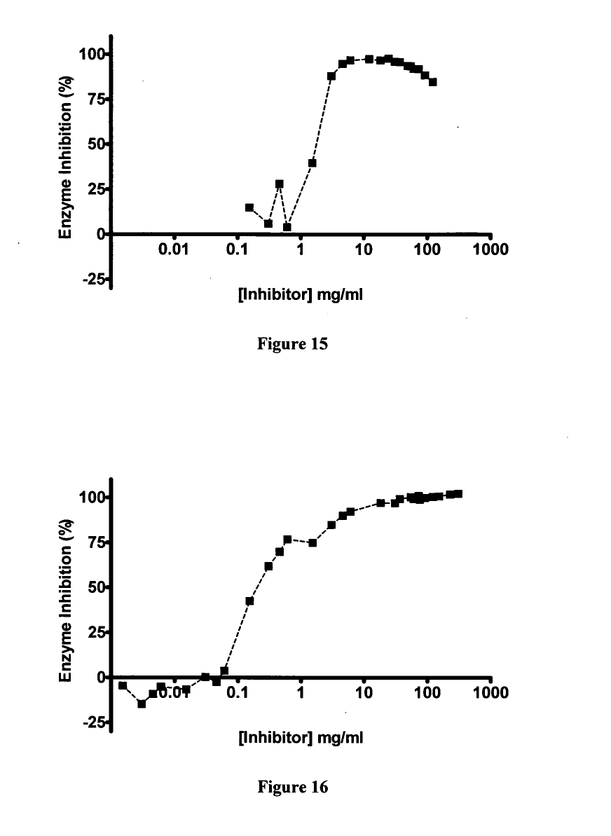

FIG. 15. Inhibition of the proteolytic activity of human gastric juice by sodium polyphosphate (68% P₂O₅) at pH 4. Mean n=1.

FIG. 16. Inhibition of snake venom metalloproteinase by sodium polyphosphate (68% P₂O₅) at pH 7.2. Mean n=1.

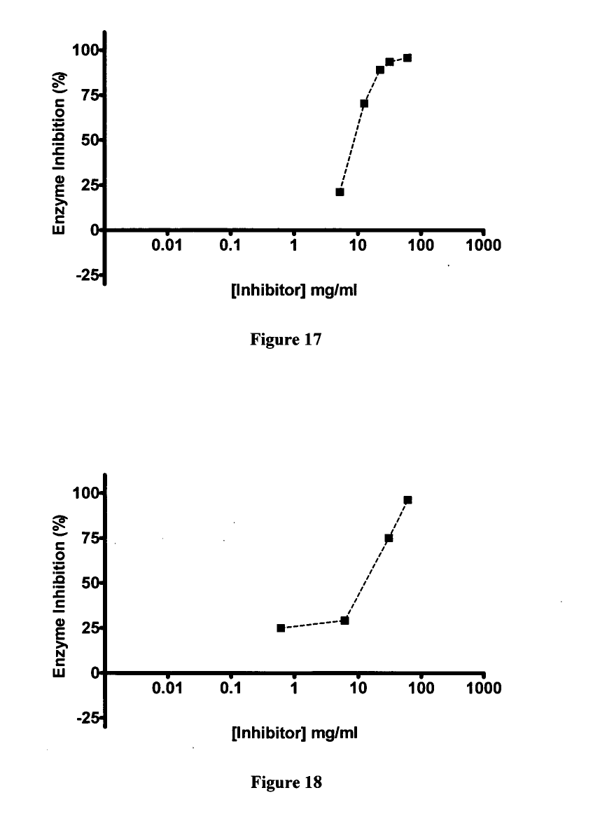

FIG. 17. Inhibition of lysozyme by sodium polyphosphate (68% P₂O₅) at pH 7. Mean n=1.

FIG. 18. Inhibition of chymotrypsin by sodium polyphosphate (68% P₂O₅) at pH 7.8. Mean n=1.

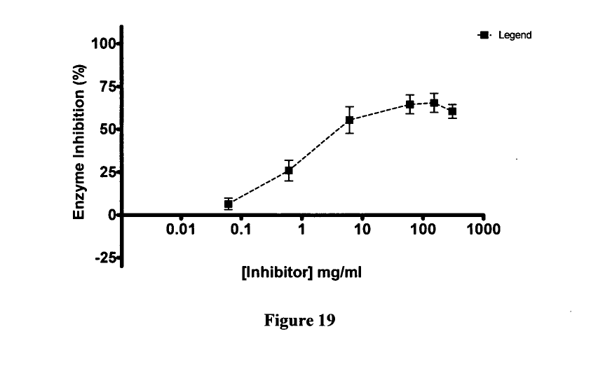

FIG. 19. Inhibition of the proteolytic activity of human chronic would by sodium polyphosphate (68% P₂O₅) at pH 7. Mean n=4±SEM.

### 1. INHIBITION OF PEPSIN

FIG. 1 shows that sodium polyphosphate (68% P₂O₅ content) inhibited the proteolytic activity of pepsin in a concentration dependent manner. The inhibition occurred across a wide range of polyphosphate concentrations. Inhibition was also pH dependent and found to be greatest at pH 4. At pH 4, levels of inhibition in excess of 90% were achieved with polyphosphate concentrations greater than 4.59 mg/ml.

FIG. 2 illustrates that the potency of inhibition varied with the polymeric chain length of sodium polyphosphates (expressed as P₂O₅ content). Sodium polyphosphates with a greater P₂O₅ content were more effective inhibitors of pepsin.

FIG. 3 demonstrates that the concentration dependent inhibition of pepsin using a polyphosphate was unaffected by the choice of alkali metal counterion. Potassium polyphosphate inhibited pepsin in a comparable manner to that observed with sodium polyphosphate.

FIG. 4 shows that sodium trimetaphosphate could also inhibit pepsin across a wide concentration range; however, the potency and extent of inhibition at maximal concentrations was not as large as that observed with polyphosphates.

### 2. INHIBITION OF COLLAGENASE

FIG. 5 shows that sodium polyphosphate (68% P₂O₅ content) inhibited the proteolytic activity of collagenase in a concentration dependent manner. The inhibition occurred across a wide range of polyphosphate concentrations. Inhibition was also pH dependent and found to be greatest at pH 7. At pH 7, levels of inhibition in excess of 70% were achieved at higher polyphosphate concentrations.

FIG. 6 illustrates that the potency of inhibition varied with the polymeric chain length of sodium polyphosphates (expressed as P₂O₅ content). Sodium polyphosphates with a greater P₂O₅ content were more effective inhibitors of collagenase.

FIG. 7 demonstrates that the concentration dependent inhibition of collagenase using a polyphosphate was relatively unaffected by the choice of alkali metal counterion. Potassium polyphosphate inhibited collagenase in a comparable manner to that observed with sodium polyphosphate.

### 3. INHIBITION OF HYALURONIDASE

FIG. 8 shows that sodium polyphosphate (68% P₂O₅ content) inhibited the digestive activity of hyaluronidase in a concentration dependent manner. The inhibition occurred across a wide range of polyphosphate concentrations. Inhibition was also pH dependent and found to be greatest at pH 4.5. At pH 4.5, levels of inhibition in excess of 90% were achieved at polyphosphate concentrations greater than 0.612 mg/ml.

FIG. 9 illustrates that the potency of inhibition varied with the polymeric chain length of sodium polyphosphates (expressed as P₂O₅ content). Sodium polyphosphates with a greater P₂O₅ content were more effective inhibitors of hyaluronidase.

FIG. 10 demonstrates that the concentration dependent inhibition of hyaluronidase using a polyphosphate was unaffected by the choice of alkali metal counterion. Potassium polyphosphate inhibited hyaluronidase in a comparable manner to that observed with sodium polyphosphate.

### 4. INHIBITION OF ELASTASE

FIG. 11 shows that sodium polyphosphate (68% P₂O₅ content) inhibited the digestive activity of elastase in a concentration dependent manner. The inhibition occurred across a wide range of polyphosphate concentrations. At high polyphosphate concentrations levels of inhibition in excess of 90% were achieved.

FIG. 12 illustrates that the potency of inhibition varied with the polymeric chain length of sodium polyphosphates (expressed as P₂O₅ content). Although sodium polyphosphates with a P₂O₅ content between 60-68% gave similar levels of inhibition, sodium polyphosphate containing 70% P₂O₅ was able to inhibit 100% of elastase activity at higher concentrations.

FIG. 13 demonstrates that the concentration dependent inhibition of elastase using a polyphosphate was not adversely affected by the choice of alkali metal counterion. Potassium polyphosphate inhibited elastase in a slightly superior manner to that observed with sodium polyphosphate.

FIG. 14 shows that sodium trimetaphosphate could also inhibit elastase across a wide concentration range; however, the extent of inhibition at maximal concentrations was not as large as that observed with polyphosphates.

### 5. INHIBITION OF OTHER ENZYME AND HUMAN BODILY FLUIDS CONTAINING ENZYMES

Sodium polyphosphate (68% P₂O₅) was also shown to inhibit other enzymes in the same classes to those described earlier.

FIG. 15 shows that polyphosphate inhibited in a concentration dependent manner the proteolytic activity of human gastric juice. At polyphosphate concentrations greater than 6.12 mg/ml nearly 100% of the proteolytic activity of human gastric juice was inhibited. Human gastric juice contains a mixture of aspartic proteases including Pepsin 1, 3a, 3b, 3c and gastricsin. The inhibition of activity for gastric juice demonstrates that polyphosphates can inhibit other aspartic proteases in addition to pepsin.

FIG. 16 illustrates that polyphosphate can inhibit in a concentration dependent manner snake venom metalloproteinase. This demonstrates that, in addition to collagenase, polyphosphate can inhibit other matrix metalloproteinases.

FIG. 17 shows that polyphosphate can inhibit in a concentration dependent manner the digestive activity of lysozyme. This demonstrates that, in addition to hyaluronidase, polyphosphate can inhibit other enzymes from the glycoside hydrolase class.

FIG. 18 shows that polyphosphate can inhibit in a concentration dependent manner the digestive activity of a-chymotrypsin. This demonstrates that, in addition to elastase, polyphosphate can inhibit other enzymes from the serine protease class.

Finally, FIG. 19 shows that polyphosphate can inhibit in a concentration dependent manner the proteolytic activity of fluid extracted from human chronic wounds.

1. Use of an unbound polyphosphate to promote chronic wound healing, in which the polyphosphate has at least 3 phosphate units.

2. Use according to claim 1, wherein the polyphosphate inhibits the action of at least one enzyme which contributes to the prevention or delay of wound healing.

3. Use according to claim 2, wherein the enzyme is one or more of pepsin, collagenase, elastase and hyaluronidase.

4. Use according to claim 1, wherein the polyphosphate has an average of from 4 to 40 phosphate units.

5. Use according to claim 1, the polyphosphate has a P₂O₅ content of at least 55% by weight.

6. Use according to claim 5, wherein the polyphosphate has a P₂O₅ content of at least 60% by weight.

7. Use according to claim 6, wherein the polyphosphate has a P₂O₅ content of from 60 to 75% by weight.

8. Use according to claim 7, wherein the polyphosphate has a P₂O₅ content of from 65 to 70% by weight.

9. Use according to claim 1, wherein the polyphosphate is an alkali metal salt.

10. Use according to claim 9, wherein the polyphosphate is a sodium or potassium salt, or a mixture thereof.

11. A method of promoting chronic wound healing comprising the administration of a therapeutically effective amount of a polyphosphate having at least 3 phosphate units.

12. A method according to claim 11, wherein the amount of polyphosphate is from 300 mg to 24,000 mg as a daily dosage.

13. A method according to claim 11, wherein the polyphosphate is administered at a pH of from 6 to 8.

14. A pharmaceutical composition comprising a therapeutically effective amount of a polyphosphate having at least 3 phosphate units in admixture with a pharmaceutically acceptable adjuvant, diluent or carrier.
